(19) United States
(12) Patent Application Publication
(10) Pub. No.: US 2009/0263892 A1
(43) Pub. Date: Oct. 22, 2009

(54) Float valve for cell culture vessel

(76) Inventors: Henry Joseph Cattadoris, Scarborough, ME (US); Gregory Roger Martin, Acton, ME (US); Allison Jean Tanner, Portsmouth, NH (US)

Correspondence Address: CORNING INCORPORATED, SP-TI-3-1, CORNING, NY, 14831, US

(21) Appl. No.: 12/148,322

(22) Filed: Apr. 18, 2008

(51) Int. Cl. C12M 1/00; F16K 27/00

(52) U.S. Cl. 435/289.1; 251/366

(57) ABSTRACT

A cell culture vessel includes a housing defining a reservoir, an inlet and an outlet in fluid communication with the reservoir, a valve assembly, and an antimicrobial filter. The valve assembly includes (i) a side wall defining a passageway for fluid flow between the reservoir and the outlet, (ii) a floating element disposed in the passageway and configured to float on the culture medium, (iii) a stop feature configured to sealingly engage the floating element to prevent the culture medium from flowing from the reservoir through the outlet, and (iv) a capture feature configured to retain the floating element within the passageway and to allow fluid to flow from the reservoir through the passageway. The microbial filter is positioned such that air flowing in the outlet to the passageway passes through the filter.

### FIELD

The present disclosure relates to vessels for culturing cells and to valves for use in cell culture vessels, particularly to valves for venting during filling.

### BACKGROUND

Air handling in cell culture vessels with little or no headspace is problematic. As such, air displaced by the culture medium needs to be vented in vessels intended to be filled completely or nearly completely with liquid culture medium. In vessels with sufficient headspace, the displaced air can be vented through a valveless filter. However, such a traditional vent would be ineffective in limited headspace vessels as the filter would become wet and inoperable.

### BRIEF SUMMARY

The present disclosure provides a vent assembly for cell culture systems. The vent assembly is effective in limited headspace systems designed to be completely or nearly completely filled with cell culture medium.

In an embodiment, the disclosure describes a cell culture vessel. The vessel includes a housing defining a reservoir, an inlet and an outlet in fluid communication with the reservoir, a valve assembly, and an antimicrobial filter. The valve assembly includes (i) a side wall defining a passageway for fluid flow between the reservoir and the outlet, (ii) a floating element disposed in the passageway and configured to float on the culture medium, (iii) a stop feature configured to sealingly engage the floating element to prevent the culture medium from flowing from the reservoir through the outlet, and (iv) a capture feature configured to retain the floating element within the passageway and to allow fluid to flow from the reservoir through the passageway. The microbial filter is positioned such that air flowing in the outlet to the passageway passes through the filter.

In an embodiment, the disclosure describes a valve assembly for allowing air to escape a reservoir of a cell culture vessel via an outlet and for preventing liquid culture medium from escaping the reservoir via the outlet. The valve assembly includes a side wall defining a passageway for fluid flow between the reservoir and the outlet. The side wall is configured to engage an opening of a cell culture vessel. The opening defines the outlet and is in fluid, communication with the reservoir. The valve assembly further includes (i) a floating element disposed in the passageway and configured to float on the culture medium, (ii) a stop feature configured to retain the floating element within the passageway and configured to operate with the floating element to prevent the culture medium from flowing from the reservoir through the outlet, and (iii) a capture feature configured to retain the floating element within the passageway and to allow fluid to flow from the reservoir through the passageway.

By allowing air to escape a reservoir of a cell culture vessel via an outlet and preventing liquid culture medium from escaping the reservoir via the outlet, the vessels and valve assemblies described herein may, in various embodiments, address some of the air handling problems associated with vessels with limited headspace. This and other advantages will be readily understood from the following detailed description when read in conjunction with the accompanying drawings.

### BRIEF DESCRIPTION OF THE DRAWINGS

FIGS. 1A-B are schematic diagrams of perspective views of cell culture vessels having an inlet and an outlet.

FIG. 2 is a schematic block diagram of a cell culture vessel having an inlet, outlet, reservoir and valve assembly.

FIGS. 3A-B are a schematic diagram of a cross section of a valve assembly in an open (A) and closed (B) configuration.

FIGS. 3C-D are bottom-up and top-down views of the valve assembly shown in FIG. 3A viewed along lines 3c and 3d, respectively.

FIGS. 4A-D are a schematic diagrams of cross sections of a valve assembly in open (A, C, D) and closed (B) configurations. FIGS. 4C-D show alternative placement of a filter.

FIG. 4E is a schematic diagram of a cross section of a valve assembly having components formed by a housing of a cell culture vessel.

FIG. 4F is bottom-up view of the valve assembly shown in FIG. 4A viewed along line 4f.

FIGS. 5A-B are a schematic diagram of a cross section of a valve assembly in an open (A) and closed (B) configuration.

FIG. 5C is bottom-up view of the valve assembly shown in FIG. 5A viewed along line 5c.

FIG. 5D is a schematic diagram of a cross section of a valve assembly having components formed by a housing of a cell culture vessel.

FIG. 5E is a schematic diagram of a cross section of an inlet portion of a cell culture vessel configured to receive a valve assembly, such as an assembly depicted in FIG. 5A.

FIG. 6 is a schematic drawing of an exploded view of a valve assembly.

The drawings are not necessarily to scale. Like numbers used in the figures refer to like components, steps and the like. However, it will be understood that the use of a number to refer to a component in a given figure is not intended to limit the component in another figure labeled with the same number. In addition, the use of different numbers to refer to components is not intended to indicate that the different numbered components cannot be the same or similar.

### DETAILED DESCRIPTION

In the following detailed description, reference is made to the accompanying drawings that form a part hereof, and in which are shown by way of illustration several specific embodiments of devices, systems and methods. It is to be understood that other embodiments are contemplated and may be made without departing from the scope or spirit of the present disclosure. The following detailed description, therefore, is not to be taken in a limiting sense.

All scientific and technical terms used herein have meanings commonly used in the art unless otherwise specified. The definitions provided herein are to facilitate understanding of certain terms used frequently herein and are not meant to limit the scope of the present disclosure.

As used in this specification and the appended claims, the singular forms “a”, “an”, and “the” encompass embodiments having plural referents, unless the content clearly dictates otherwise. As used in this specification and the appended claims, the term “or” is generally employed in its sense including “and/or” unless the content clearly dictates otherwise.

As used herein, “have”, “having”, “include”, “including”, “comprise”, “comprising” or the like are used in their open ended sense, and generally mean “including, but not limited to”.

The present disclosure describes, inter alia, valve assemblies or cell culture vessels having valve assemblies that may be used in cell culture environments with limited head space. The valve assemblies described herein, in various embodiments, allow for release of gas and vapor from a reservoir of a cell culture vessel while the vessel is being filled with culture medium and prevent culture medium from leaking as the vessel is filled.

Nearly any cell culture vessel can be adapted for use with the valve assemblies. Examples of suitable cell culture vessels for use with the valve assemblies or vent caps described herein include jars, flasks, beakers, roller bottles, tubes, perfusion chambers, bioreactors, and fermenters. Some commercially available cell culture vessels that may be readily adapted to include a vent assembly or vent cap as described herein include the PETAKA™ Cell Culture vessel, (Celartia, Ltd.), CELL STACK™ culture chambers (Corning, Inc.), HYPERFLASK™ cell culture chambers (Corning, Inc.), ROBOFLASK™ cell culture chambers (Corning, Inc.) and OPTICELL™ cell culture systems (Nunc International). The greatest benefit will likely be achieved by cell culture vessels with little or no headspace and with a single fluid entrance port.

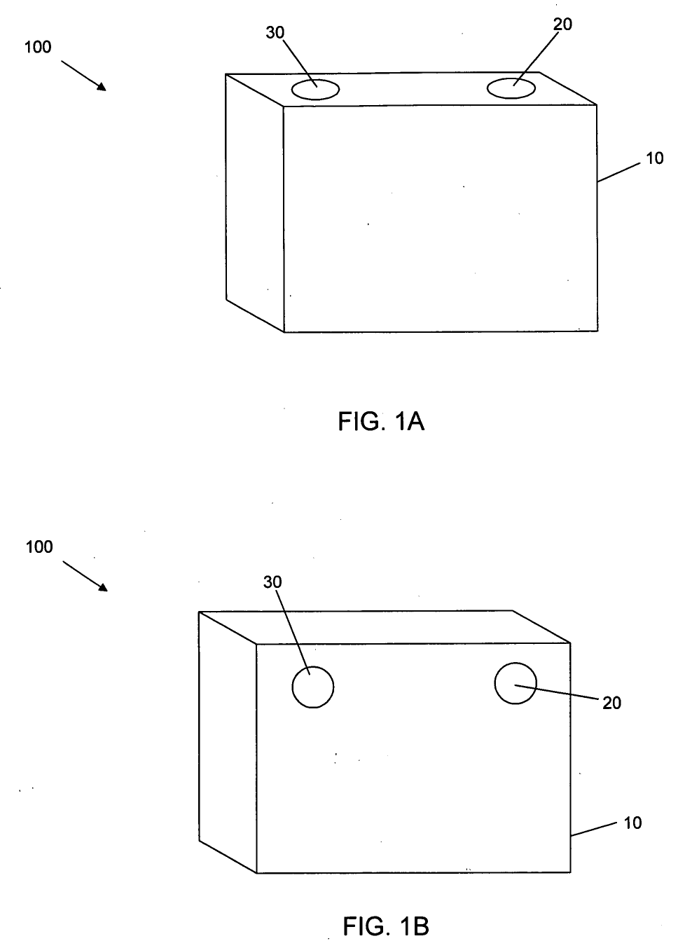

Referring to FIGS. 1A-B, perspective views of cell culture vessels 100 are shown. In the depicted embodiment, the vessels 100 have an inlet 20 and an outlet 30 defined by housing 10. The inlet 20 and outlet 30 are in fluid communication with a cell culture chamber, such as an internal reservoir defined by housing 10, of the vessel 100. The chamber or reservoir may be filled with cell culture medium, cells, or the like via inlet 20. In various embodiments, material may also be withdrawn via inlet 20. As the internal chamber or reservoir is being filled via inlet 20, displaced air within the chamber can be vented via outlet 30.

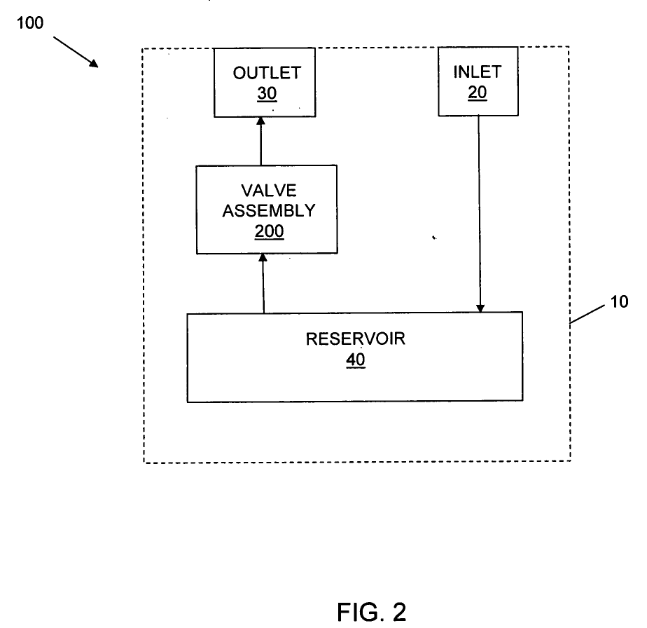

Referring now to FIG. 2, a block diagram of a representative cell culture vessel 100 is shown. The direction of the arrows in FIG. 2 are for purposes of illustration of a representative fluid (gas, vapor, or liquid) flow path. However, it will be understood that fluid may flow in the opposite direction shown. As fluid is introduced via inlet 20, reservoir 40 begins to fill, displacing fluid through valve assembly 200 and out outlet 30. The valve assembly 200 is configured to allow air to escape as the reservoir 40 is being filled, but to prevent liquid, such as culture medium, to escape when the reservoir 40 is full.

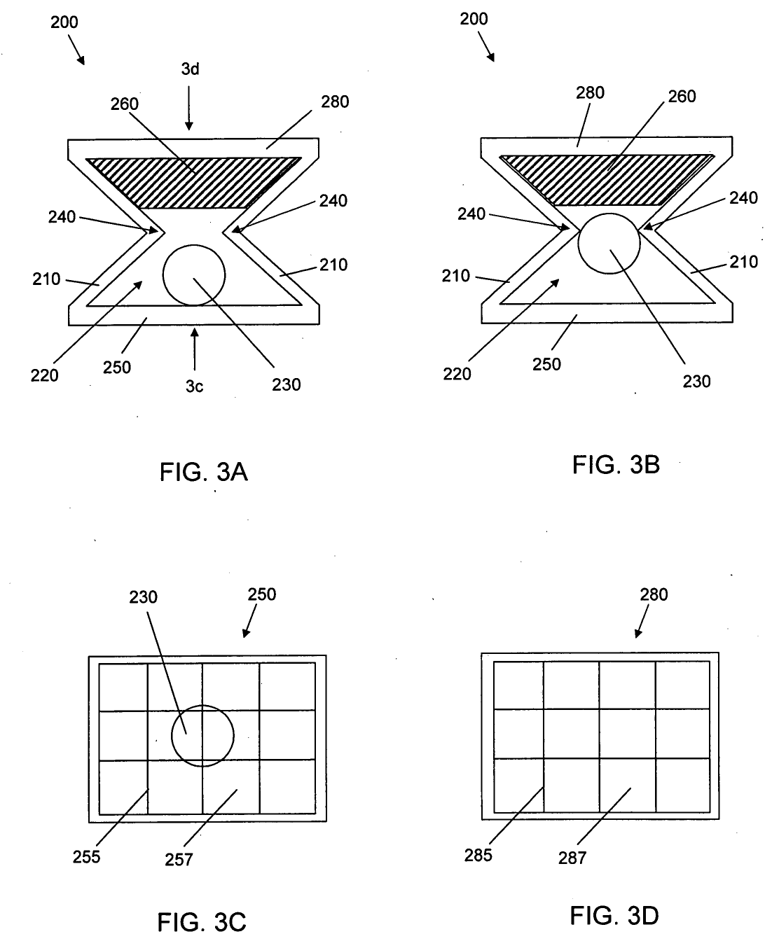

In FIG. 3 an embodiment of a representative valve assembly 200 is shown. The valve assembly 200 includes a side wall 210 defining at least a portion of a passageway 220 for fluid flow between a reservoir and an outlet of a cell culture vessel. A floating element 230 is disposed in the passageway 220. The floating element 230 is configured to float on culture medium as culture medium enters the passageway 220 from the reservoir. The vent assembly 200 includes a stop feature 240 configured to sealingly engage the floating element 230 as the level of culture medium rises in the passageway 220 to prevent flow of culture medium beyond the stop feature 240. FIG. 3A depicts the valve assembly 200 in an open configuration, and FIG. 3B depicts the valve assembly in a closed configuration. In the depicted embodiment, the stop feature 240 protrudes into the passageway 220, which is generally hourglass shaped. As the floating element 230, which is in the form of a sphere in the depicted embodiment, floats and rises on culture medium (not shown), the floating element 230 sealing engages the stop feature 240, which includes the internal restriction of the hourglass shaped passageway 220.

The vent assembly 200 depicted in FIG. 3 also includes a capture feature 250 configured to retain the floating element 230 in the passageway 220. A bottom-up view of the capture feature 250 retaining the floating element 230 is shown in FIG. 3C, which is viewed along line 3c in FIG. 3A. The depicted capture feature 250 includes retaining elements 255 that serve to retain the floating element 230 in the passageway 220 and to allow air flow from the reservoir into the passageway 220 while the floating element 230 engages the capture feature 250. The retaining elements 255 may form a grid having openings 257 that allow for flow of fluid. Of course, any retention structure having openings for fluid flow and sized to retain the floating element 230 in the passageway 220 may be employed.

The vent assembly 200 depicted in FIG. 3 also includes a microbial filter 260 for preventing contaminated air from an outlet from entering the passageway 220 and thus the reservoir. The filter 260 is positioned distal to the stop feature 240 relative to the reservoir (not shown in FIG. 3). Such positioning is desirable because the sealing cooperation of the stop feature 240 and the floating element 230 may prevent the filter from contacting the liquid culture medium, keeping the filter dry and functional. The filter 260 may sealingly engage the side wall 210 via any suitable mechanism, such as adhesive or interference fit, to prevent contaminated air from an outlet reaching the passageway 220. The vent assembly 220 may also include a protective member 280 positioned to protect the filter 260. A top-down view of the protective member 280 is shown in FIG. 3D, which is viewed from the perspective of line 3d in FIG. 3A. The depicted protective member 280 includes elements 285 that form a grid and define openings 287 that allow for flow air. Of course, any protective structure having openings that allow for air flow may be employed.

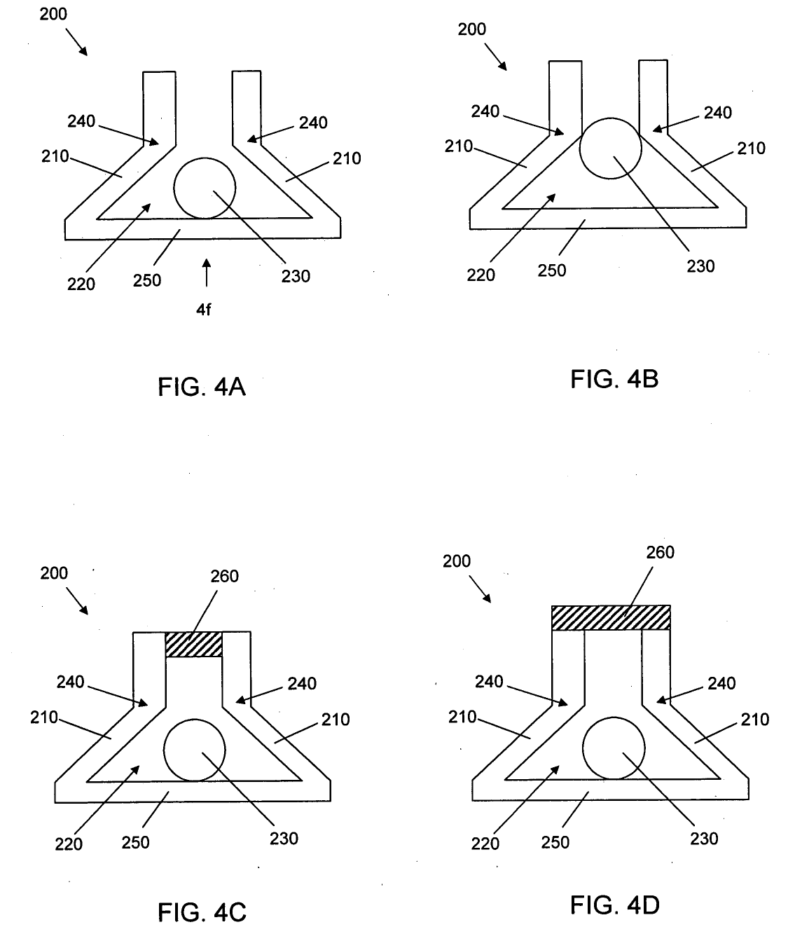

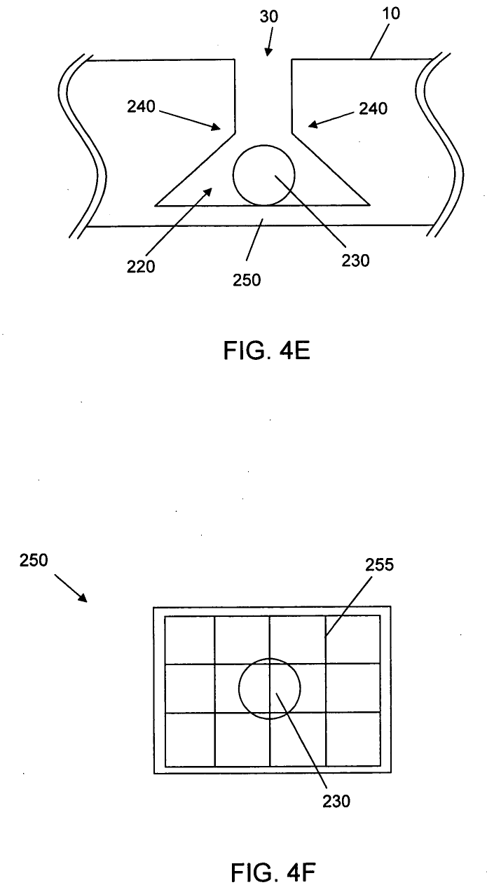

Referring now to FIG. 4, various embodiments of representative vent assemblies are shown. The vent assemblies shown in FIG. 4 are similar to those shown in FIG. 3. For example, the vent assemblies have a side wall 210 forming a passageway 220. In the embodiments depicted in FIG. 4, the passageway 220 is generally funnel shaped. Stop feature 240 is formed from the side wall 210 generally at the smaller diameter end of the frustrum of the cone of the funnel shaped passageway 220. The floating element 230 is generally spherical in the depicted embodiment and is configured to sealingly engage the stop feature 240 as the floating element 230 floats on culture medium (not shown) and rises in passageway 220. FIG. 4A depicts the valve assembly 200 in an open configuration, and FIG. 4B depicts the valve assembly 200 in a closed configuration. FIGS. 4C-D show alternative configurations for placement of filter 260. As with the embodiment depicted in FIG. 3, the filter 260 depicted in FIGS. 4C-D is positioned distal the stop feature 240 relative to the reservoir (not shown in FIG. 4). Such positioning is desirable because the sealing cooperation of the stop feature 240 and the floating element 230 may prevent the filter from contacting liquid culture medium. As with the vent assembly shown in FIG. 3, the assemblies shown in FIG. 4 include a capture feature 250 configured to retain the floating element 230 within the passageway 220 and configured to allow air to flow between the reservoir and the passageway 220. FIG. 4F shows a bottom-up view of the capture feature 250, viewed along line 4f of FIG. 4A.

Referring now to FIG. 4E, an embodiment wherein the housing 10 of a cell culture vessel (such as a vessel depicted in FIG. 1) forms the side wall of the valve assembly is shown. In other aspects, the valve assembly is similar to that shown and described with regard to FIGS. 4A-D and F.

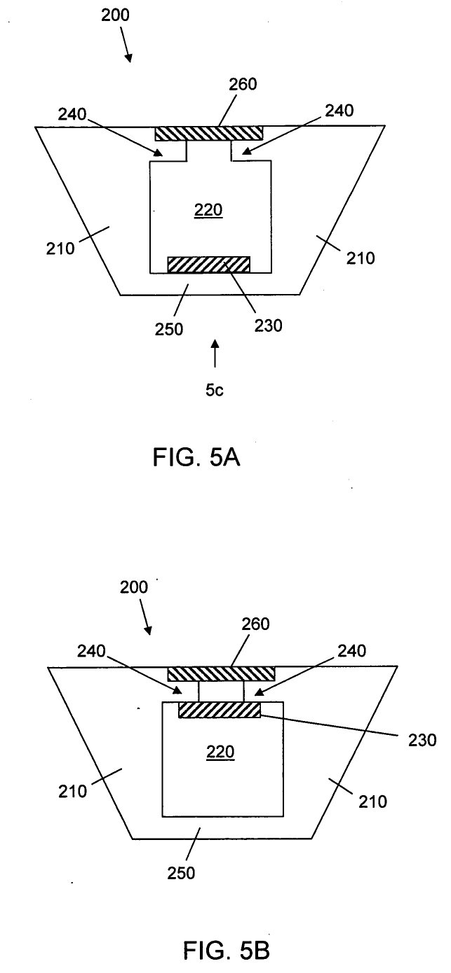

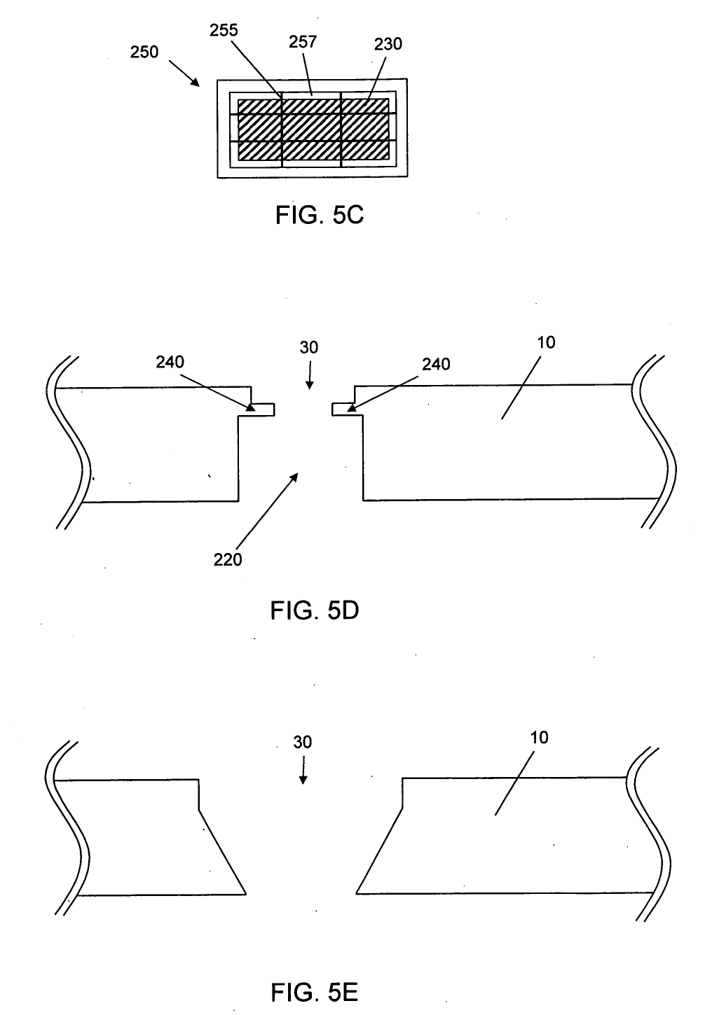

In FIG. 5 representative valve assemblies having a box shaped floating element 230 is shown. As with the valve assemblies depicted in FIGS. 3-4, the valve assembly depicted in FIG. 5A-C includes a sidewall 210 forming a passageway 220 through which fluid may flow. A capture element 250 extends across one end of the side wall 210 and is configured to retain the floating element 230 within the passageway 220. A bottom-up view of the capture element 250 is shown in FIG. 5C from the perspective of line 5c of FIG. 5A. The capture element 250 includes elements 257 forming a grid having openings 257 through which fluid may flow between a reservoir of a cell culture vessel and the passageway 220. The floating element 230 is configured to float on culture medium (not shown) and sealing engage stop feature 240 (see FIG. 5B). Stop feature 240 in the depicted embodiment extends into the passageway 220 from the side wall 210 and forms an opening of a diametric dimension smaller than that of the floating element 230. A filter may be disposed in a counter bore is formed in the stop feature 240.

FIG. 5D depicts a representative cross section of a portion of a housing 10 of a cell culture vessel, where the housing 10 forms the side wall and stop feature 240 of a valve assembly similar to that depicted in FIGS. 5A-B. The passageway 220 of the valve assembly formed by the housing 220 opens external to the housing at outlet 30.

FIG. 5E depicts a representative cross section of a portion of a housing 10 of a cell culture vessel, where the housing 10 defines an outlet 30 configured to receive a valve assembly 200 as depicted in, e.g., FIGS. 5A-B. The valve assembly 200 is configured to sealing engage the housing 10. The valve assembly may have external threads (not show) configured to engage internal threads (not shown) formed in housing 10 such that valve assembly 200 may be screwed into housing 10. The valve assembly 20 may fit into the opening formed by housing 10 via interference fit to form a seal. Of course, a seal may be formed between the opening formed in housing 10 and the side wall 210 of the valve assembly 200 through any other suitable mechanism, such as an adhesive or the like.

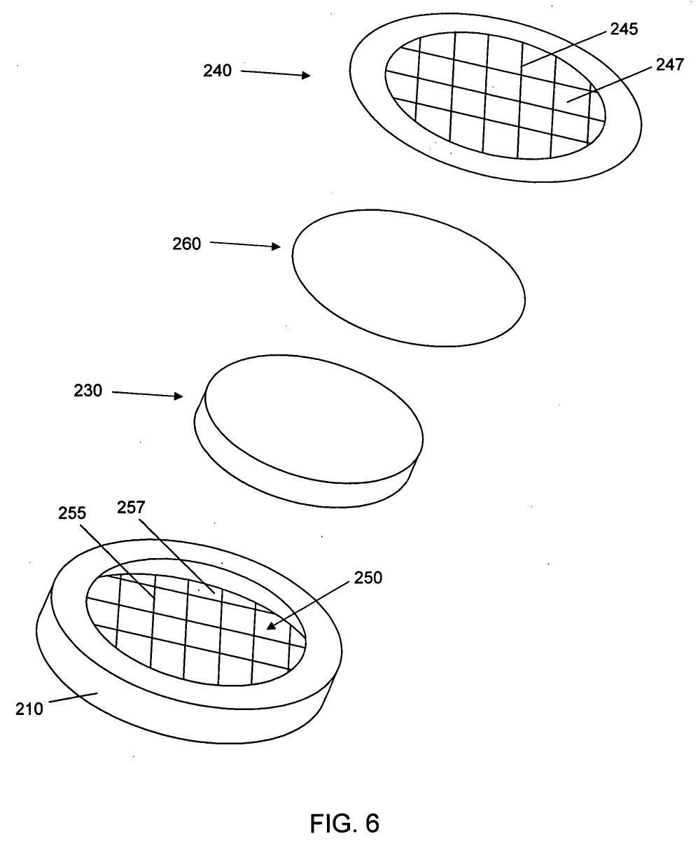

Referring now to FIG. 6, another representative valve assembly is shown. The valve assembly is configured to be inserted into an opening of a cell culture vessel such that when inserted in the opening a passageway of the valve assembly is in fluid communication with an outlet and a reservoir of the vessel. The valve assembly includes a side wall 210 defining the passageway. The valve assembly depicted in FIG. 6 also includes a floating element 230, which is disk shaped in the depicted embodiment, disposed in the passageway formed by the side wall 210. A capture element 250 is disposed across an end of the side wall 210 intended to face the reservoir of the vessel and includes elements 255 forming a grid defining openings 257 through which fluid may flow between the reservoir and the passageway. Capture element 250 retains the floating element 230 within the passageway. The valve assembly further includes a stop feature 240 that extends across an end of the side wall 210 intended to face an outlet of a cell culture vessel. The depicted stop feature 240 has elements 245 forming a grid defining openings 247 through which air may pass from the outlet into the passageway. A filter 260 is disposed in the passageway, such that air that flows through the outlet into the passageway passes through the filter. The filter 60 may be positioned such that it engages the elements 245 of the stop feature 240. The stop feature 240 may serve a similar purpose to protective member discussed above with regard to FIG. 3.

In the embodiment depicted in FIG. 6, the floating element 230 rises as culture medium fills the passageway of the vent assembly and engages the filter 60, which is in contact with the stop feature 240, to prevent further movement of the floating element 240 and to seal the passageway such that culture media in the passageway does not contact the filter 260 and does not leak out the outlet of the cell culture vessel. Accordingly, in this embodiment, the filter 60 and the stop feature 240 together act as a stop feature that the floating element 230 is configured to sealingly engage.

It will be understood that components and aspects of the various embodiments described herein may be interchanged or omitted as desired. For example, a protective member for protecting a filter as described with regard to FIG. 3 may be employed with any of the valve assemblies of FIGS. 4-5. By way of further example, a stop feature that extends from a side wall into a passageway as described with regard to FIGS. 3-5 may be employed with a valve assembly described with regard to FIG. 6.

For the various embodiments described herein, it will also be understood that various components may be integrally formed or may be formed from separate parts. For example, the stop feature described with regard to FIG. 6 may be integrally formed with the side wall or may be a separate piece attached to side wall. Regardless of whether integrally formed or formed from separate parts, components, particularly those that may come in contact with cell culture medium, are preferably made of material that is not toxic to cells being cultured.

For example, a cell culture vessel housing or a body of a vent assembly may be formed from material including a ceramic substance, glass, or plastic. Suitable glass materials include soda-lime glass, pyrex glass, vycor glass, and quartz glass. Suitable plastics or polymers include, poly(vinyl chloride), poly(methyl methacrylate), poly(dimethylsiloxane) monomethacrylate, cyclic olefin polymers, fluorocarbon polymers, polystyrenes, polyethylene, polycarbonate, polyester, polypropylene; copolymers such as poly(styrene-co-maleic anhydride), poly(ethylene-co-acrylic acid), derivatives of these or the like. Many of such materials may allow for exchange of gasses from the reservoir to outside the vessel (and vice-versa). Some of such materials can be formed to be useful for exchange of gasses for purposes of cell culture, but the rate at which gas can cross such materials may not be sufficient to vent gas during filling of a reservoir.

A floating element as described herein may be made of any suitable material capable of floating on liquid that may be introduced into a cell culture vessel. In some embodiments, the floating element is made of polymeric material such as foamed (closed cell) polypropylene, foamed polystyrene, polyethylene or polymethylpentene.

A filter as described herein preferably prevents passage of particles having an average diameter or diametric dimension of between about 0.1 and about 0.3 microns. For example, the filter may be what is typically referred to as a 0.2 micron filter. The filter may include a prefilter layer. For example, the prefilter layer may be configured to prevent the passage of particles having an average diametric dimension of between about 80 micrometers and about 120 micrometers. The filter may be formed from hydrophobic material to lower the possibility of aqueous liquid, such as culture media, from passing through the filter. For example, the filter may be formed from polytetrafluoroethylene, polyvinylidene fluoride, or polypropylene

Grid forming elements of a capture element, protective member, or stop feature as described herein may be formed from polymeric fibers, metallic fibers, or the like.

Thus, embodiments of FLOAT VALVE FOR CELL CULTURE ASSEMBLY are disclosed. One skilled in the art will appreciate that the arrays, compositions, kits and methods described herein can be practiced with embodiments other than those disclosed. The disclosed embodiments are presented for purposes of illustration and not limitation.

What is claimed is:

1. A cell culture vessel, comprising:
a housing defining a reservoir for containing a cell culture medium;
an inlet in fluid communication with the reservoir for filling the reservoir with the culture medium;
an outlet in fluid communication with the reservoir;
a valve assembly having
(i) a side wall defining at least a portion of a passageway for fluid flow between the reservoir and the outlet,
(ii) a floating element disposed in the passageway and configured to float on the culture medium,
(iii) a stop feature configured to sealingly engage the floating element to prevent the culture medium from flowing from the reservoir through the outlet, and
(iv) a capture feature configured to retain the floating element within the passageway and to allow fluid to flow from the reservoir through the passageway; and
a microbial filter positioned such that air flowing in the outlet to the passageway passes through the filter.

2. The cell culture vessel of claim 1, wherein the housing forms the side wall of the valve assembly.

3. The cell culture vessel of claim 1, wherein the capture feature is configured to allow air to flow from the reservoir through the passageway when the floating element is in contact with the capture feature.

4. The cell culture vessel of claim 1, wherein the stop feature is in the passageway.

5. The cell culture vessel of claim 4, wherein the floating element engages the stop feature to prevent the culture medium from flowing from the reservoir to the outlet.

6. The cell culture vessel of claim 1, wherein the stop feature is disposed across an end of the side wall that is distal to the reservoir.

7. The cell culture vessel of claim 6, wherein the floating element engages the filter to prevent the culture medium from flowing from the reservoir to the outlet.

8. The cell culture vessel of claim 1, wherein the stop feature is integrally formed with the side wall.

9. The cell culture vessel of claim 1, wherein the stop feature comprises the filter.

10. The cell culture vessel of claim 1, wherein the filter is positioned distal the stop feature relative to the reservoir.

11. A valve assembly for allowing air to escape a reservoir of a cell culture vessel via an outlet and for preventing liquid culture medium from escaping the reservoir via the outlet, the valve assembly comprising:
(i) a side wall defining a passageway for fluid flow between the reservoir and the outlet and configured to engage an opening of a cell culture vessel, wherein the opening defines the outlet and is in fluid communication with the reservoir,
(ii) a floating element disposed in the passageway and configured to float on the culture medium,
(iii) a stop feature configured to retain the floating element within the passageway and configured to operate with the floating element to prevent the culture medium from flowing from the reservoir through the outlet, and
(iv) a capture feature configured to retain the floating element within the passageway and to allow fluid to flow from the reservoir through the passageway.

12. The valve assembly of claim 11, further comprising a microbial filter positioned such that air flowing from external the housing via the outlet into the passageway passes through the filter.

13. The valve assembly of claim 12, wherein the filter is positioned distal the stop feature relative to the reservoir to filter air flowing from the outlet into the passageway.

14. The valve assembly of claim 12, wherein the stop feature comprises the filter.

15. The valve assembly of claim 11, wherein the capture feature is configured to allow air to flow from the reservoir through the passageway when the floating element is in contact with the capture feature.

16. The valve assembly of claim 11, wherein the stop feature is disposed about the opening.

17. The valve assembly of claim 16, further comprising a filter positioned such that air flowing from external the housing via the outlet into the passageway passes through the filter, wherein the floating element engages the filter to prevent the culture medium from flowing from the reservoir to the outlet.
